(19) United States
(12) Patent Application Publication
(10) Pub. No.: US 2020/0222530 A1
(43) Pub. Date: Jul. 16, 2020

(54) PREVENTION OF ALLERGY

(71) Applicant: ASIT BIOTECH S.A., Bruxelles (BE)

(72) Inventors: Thierry LEGON, Roosbeek (BE); Gaetan THIRION, Purnode (BE)

(21) Appl. No.: 16/338,672

(22) PCT Filed: Oct. 5, 2017
(86) PCT No.: PCT/EP2017/075398

§ 371 Date: Apr. 1, 2019

(30) Foreign Application Priority Data
Oct. 5, 2016 (EP) 16192425.3
May 16, 2017 (EP) 17171268.0

(51) Int. Cl. A61K 39/35; A61K 39/36; A61P 37/08

(52) U.S. Cl. CPC A61K 39/35; A61K 2039/545; A61P 37/08; A61K 39/36

(57) ABSTRACT

A preparation comprising peptides from natural allergens for use in the prevention of development of allergy.

The present invention relates to preparations for preventing the development of an allergy and methods and uses related thereto.

Allergies are based on a hypersensitivity of the immune system to something in the environment that usually causes little or no problem for most people. It comprises hay fever, food allergies, atopic dermatitis, allergic asthma and anaphylaxis. Allergies are common in the developed world.

Common allergens are pollens, house dust mites, moulds, drugs, foods and animal hair and dander.

Allergic diseases have a strong genetic relation. Allergic parents are more likely to have allergic children. It seems that the likelihood of developing allergies is inherited.

The most common allergy diseases are rhinitis, asthma and atopic dermatitis. Allergic asthma is a chronic inflammatory disorder. Symptomatic treatment of allergic disorders comprises administration of antihistamines, ß-antagonists and corticosteroids.

Furthermore, the so called “specific” immunotherapy is based on a hyposensitization. Typically, patients with confirmed allergy are administered with subcutaneous injection of the specific offending allergens. Treatment is started with small allergen doses and the doses are increased. Treatment is typically maintained for several years. This type of treatment suffers from poor patient compliance and has been questioned due to safety reasons because a patient can suffer from severe anaphylactic reactions.

In addition to methods comprising repeated subcutaneous injections there are also oral hyposensitization methods.

U.S. Pat. No. 4,822,611 discloses a method for treating existing allergies comprising oral treatment with allergens. It describes the use of commercially available “bulk” allergenic extracts showing batch-to-batch variation and differences in extracts from different manufactures. The preparation of these extracts is not described.

GB 1 247 614 discloses a method of extracting an allergen. The aim of this method is to have a more complete and effective allergenic extract by including all extractable components of the allergen.

U.S. Pat. No. 5,770,698 discloses a process for purifying extracts of allergenically active proteins. The spectrum of FIG. 2 of US '698 does not present a peak at 280 nm. This implies that the extract contains significant amount on non-protein impurities.

WO 99/22762 discloses a similar method; therefore, the product comprises large amounts of non-protein impurities, too.

U.S. Pat. No. 6,312,711 discloses a pharmaceutically or food composition intended for treating pathologies associated with graft rejection or allergic autoimmune reaction comprising the administration of a complex of a stress protein and epitopes of an antigenic structure.

WO 2013/011095 discloses a pharmaceutical preparation for subcutaneous injection comprising between 0.5 ng and 200 μg of HSP70 between 0.5 and 100 μg of fragments of an antigenic structure.

Despite strong efforts in the development of treatment methods, it is still not sufficiently addressed. There is still a need for improved treatments of allergies.

Up to now, there has been no prophylactic treatment except avoidance of exposure to allergens.

Surprisingly, it has been found that it is possible to make a prophylactic treatment to avoid development of allergies.

One embodiment is a preparation comprising peptides from a natural allergen for use in the prevention of allergy. These peptides are for example obtained from natural allergens by hydrolysis.

The present invention uses peptides of allergens to prevent development of allergy by administering small amounts of the peptides to prevent a later development of an allergy.

According to prior art, a patient suffering from allergy is treated with a drug to supress the symptoms of allergy or by hyposensitization which may reduce the patient reactions to allergens.

In contrast to these methods the present invention is intended to prevent development of the allergy in a patient that is at a risk of developing an allergy.

Therefore, the subject matter of the present invention is a preparation comprising peptides of a natural allergen for use in the prevention of allergy or more specifically for the prevention of development of allergy.

Subject matter of the invention is a preparation comprising peptides from natural allergens for use in the prevention of development of allergy.

In other words, the invention provides a method by which a tolerogenic or positive memory effect can be induced towards a compound that is not yet allergenic but could become allergenic in a patient at risk.

Preferably the preparation is administered 2 to 10 times, preferably at intervals of 2 to 10 days, for example once per week.

The preparation may be administered in constant amounts or increasing amounts.

Preferably, the preparation is free of immune stimulating adjuvants.

For example, if one or both of the parents are suffering from peanut allergy there is an increased risk that the children develop peanut allergy as well. Peanut allergy may be life threatening.

According to the method of the invention, a child could be treated prior to any contact with peanut to develop a status of the immune system that prevents development of the allergy to peanut.

In one embodiment, the peptides of an allergen are hydrolyzed allergen peptides.

In one embodiment, said hydrolyzed allergen peptides are obtainable by
a) extracting a natural source of allergens comprising allergenic proteins to form an extract,
b) purifying of said extract to remove non-protein components to form a purified extract,
c) denaturing said purified extract to form a purified denatured extract,
d) hydrolysing the purified denatured extract to form hydrolysed allergen peptides.

In a further embodiment, said hydrolyzed allergen peptides are obtainable by
a) extracting a source of allergens comprising allergenic proteins to form an extract,
b) purifying the extract to remove non-protein components to form a purified extract,
c) denaturing the purified extract with a first denaturing agent to form a purified denatured extract,
d) refining the purified denatured extract to remove impurities to form a refined denatured extract,
e) denaturing the refined denatured extract with a second denaturing agent to form denatured allergen mixture, and
f) hydrolyzing the denatured allergen mixture to form the hydrolyzed allergen peptides.

Preferred allergens are selected among pollen allergens, milk allergens, venom allergens, egg allergens, weed allergens, grass allergens, tree allergens, shrub allergens, flower allergens, vegetable allergens, grain allergens, fungi allergens, fruit allergens, berry allergens, nut allergens, seed allergens, bean allergens, fish allergens, shellfish allergens, seafood allergens, meat allergens, spices allergens, insect allergens, mite allergens, mould allergens, animal allergens, pigeon tick allergens, worm allergens, soft coral allergens, animal dander allergens, nematode allergens, allergens of Hevea brasiliensis.

Preferably, natural allergens are used and hydrolyzed, although recombinant preparations might be suitable as well.

In one embodiment, denaturing is performed with a denaturing agent selected from the group of chaotropic agents, reducing agents and mixtures thereof, preferably among urea, guanidinium chloride, dithiotreitol, thioglycerol, β-mercaptoethanol, TCEP (tris (2-carboxyethyl) phosphine) and mixtures thereof.

In one embodiment, the hydrolysis is performed with an enzyme, preferably pepsin, trypsin or chymotrypsin, more preferably wherein hydrolyzing is performed in the presence of a chaotropic agent, preferably selected from urea and guanidinium chloride and reducing reagent preferably from TCEP or DTT.

In one embodiment, the method further comprises purifying the hydrolyzed allergens to remove peptides with molecular weights above 10.000 Da and below 1.000 Da, wherein 70%, more preferably 80% of the peptides are between 10.000 Da and 1.000 Da.

A further embodiment is the use of the preparation as an allergy vaccine.

A further embodiment is a method of preventing allergy comprising administering the preparation of the invention to a patient at risk of developing allergy.

WO 2008/000783 describes a method of purifying allergens overcoming at least some of the drawbacks of prior art, especially to provide antigens from natural allergens with a significant reduced capability to trigger allergenicity reaction compared to the crude allergen extract but able to stimulate T-cells as well.

WO 2012/172037 discloses a method for the production of hydrolyzed allergens.

The disclosure of WO 2008/000783 and WO 2012/172037 is incorporated in its entirety into this application.

All references cited herein are incorporated by reference to the full extent to which the incorporation is not inconsistent with the express teachings herein.

The effects of the present invention are exemplified as follow:

### EXAMPLE 1—PEANUT

### 1. Aim of the Study

Evaluation of the induction of a possible “memory effect” in Balb/c mice treated twice per week during 3 weeks with 400 μg of peanuts peptides (ARA/PEP_SOL UBT13J03) in mannitol-trehalose environment (the injections were performed sub-cutaneously without any adjuvant) and challenged by a unique intraperitoneal (ip) injection of 100 μg of peanut native proteins without any adjuvant.

### 2. Summary of the Protocol

| | Group 1 | | Group 2 | | Group 3 | | Group 4 | | Group 5 | | Group 6 | |
|---|---|---|---|---|---|---|---|---|---|---|---|---|
| Number of animals | 10 | | 10 | | 10 | | 10 | | 10 | | 10 | |
| Treatment: | ARA/ | | ARA/ | | ARA/ | | ARA/ | | ARA/ | | PLACEBO | |
| Antigens in M/T | PEP_SOL | | PEP_SOL | | PEP_SOL | | PEP_SOL | | PEP_SOL | | | |
| | 13J03 | | 13J03 | | 13J03 | | 13J03 | | 13J03 | | | |
| Injected dose | 400 | μg | 400 | μg | 400 | μg | 400 | μg | 400 | μg | 400 | μg |
| (treatment) | | | | | | | | | | | | |
| Way of | SC | | SC | | SC | | SC | | SC | | SC | |
| administration | | | | | | | | | | | | |
| (treatment) | | | | | | | | | | | | |
| Injected volume | 300 | μl | 300 | μl | 300 | μl | 300 | μl | 300 | μl | 300 | μl |
| (treatment) | | | | | | | | | | | | |
| Injections | J0-4-7- | | J0-4-7- | | J0-4-7- | | J0-4-7- | | J0-4-7- | | J0-4-7- | |
| calendar | 11-14 | | 11-14 | | 11-14 | | 11-14 | | 11-14 | | 11-14 | |
| (treatment) | et 18 | | et 18 | | et 18 | | et 18 | | et 18 | | et 18 | |
| Challenge | ARA/ | | ARA/ | | ARA/L | | ARA/ | | ARA/ | | ARA/ | |
| | ALL_SOL | | ALL_SOL | | ALL_SO | | ALL_SOL | | ALL_SOL | | ALL_SOL | |
| | NATIVES | | NATIVES | | NATIVES | | NATIVES | | NATIVES | | NATIVES | |
| | 13I12 | | 13I12 | | 13I12 | | 13I12 | | 13I12 | | 13I12 | |
| Injected dose | 100 | μg | 100 | μg | 100 | μg | 100 | μg | 100 | μg | 100 | μg |
| (challenge) | | | | | | | | | | | | |
| Way of | IP | | IP | | IP | | IP | | IP | | IP | |
| administration | | | | | | | | | | | | |
| (challenge) | | | | | | | | | | | | |
| Injected volume | 200 | μl | 200 | μl | 200 | μl | 200 | μl | 200 | μl | 200 | μl |
| (challenge) | | | | | | | | | | | | |

| | | | | | | |
|---|---|---|---|---|---|---|
| Injection | J28 | J35 | J42 | J49 | J56 | J28 |
| calendar | | | | | | |
| (challenge) | | | | | | |

### 3. Denomination of the Groups

| | |
|---|---|
| Group 1 | 13J03-DP/Challenge J28 |
| Group 2 | 13J03-DP/Challenge J35 |
| Group 3 | 13J03-DP/Challenge J42 |
| Group 4 | 13J03-DP/Challenge J49 |
| Group 5 | 13J03-DP/Challenge J56 |
| Group 6 | PLACEBO (M/T)/Challenge J28 |

### 4. Correspondence Between Days Post-Beginning of the Treatment and Days Post-Challenge

All the groups have the same calendar for the treatment phase till the Day 28

| Groups | Days post-beginning of the treatment | Days Post-Challenge |
|---|---|---|
| 1 and 6 | 28-35-42-49-56-63-70 | 0-7-14-21-28-35-42 |
| 2 | 35-42-49-56-63-70 | 0-7-14-21-28-35 |
| 3 | 42-49-56-63-70 | 0-7-14-21-28 |
| 4 | 49-56-63-70 | 0-7-14-21 |
| 5 | 56-63-70 | 0-7-14 |

Caution:

ARA/PEP_SOL is commonly used for peanut peptides.

ARA/ALL_SOL is commonly used for peanut proteins

### 5. Results

Here are the results obtained by ELISA for the production of specific IgG anti-ARA proteins (native—native proteins coating)

a. Evolution of IgG Production Before any Challenge (from Day 0 to Day 28):

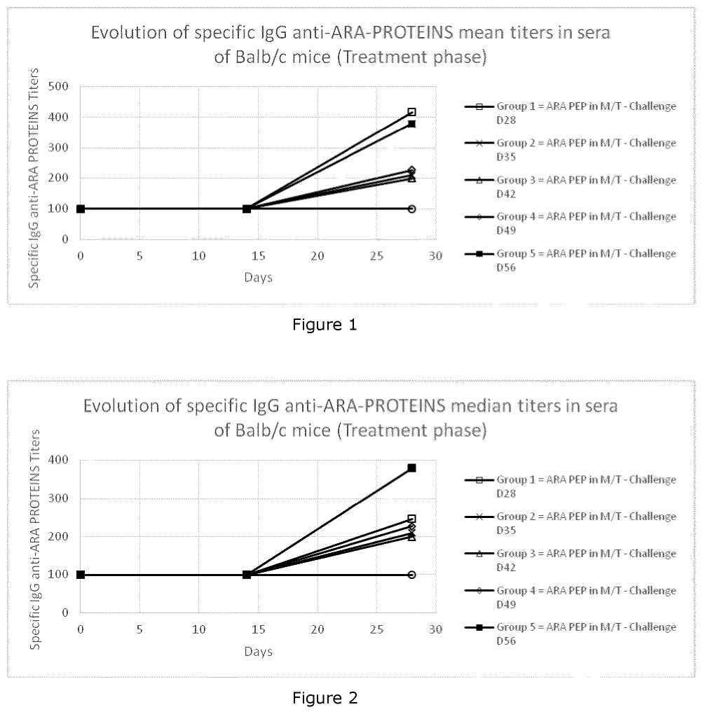

FIGS. 1 and 2 show that the treatment of the mice with peanut peptides as drug product leads to a very weak production of specific IgG anti-ARA PROTEINS till the Day 28. The mice treated with PLACEBO show no specific IgG anti-ARA PROTEINS production.

b. Evolution of IgG Production after the Challenge (from the Challenge Day to the Day 42 Post-Challenge): Caution: Not all Groups Reach this Day 42 Post-Challenge!!

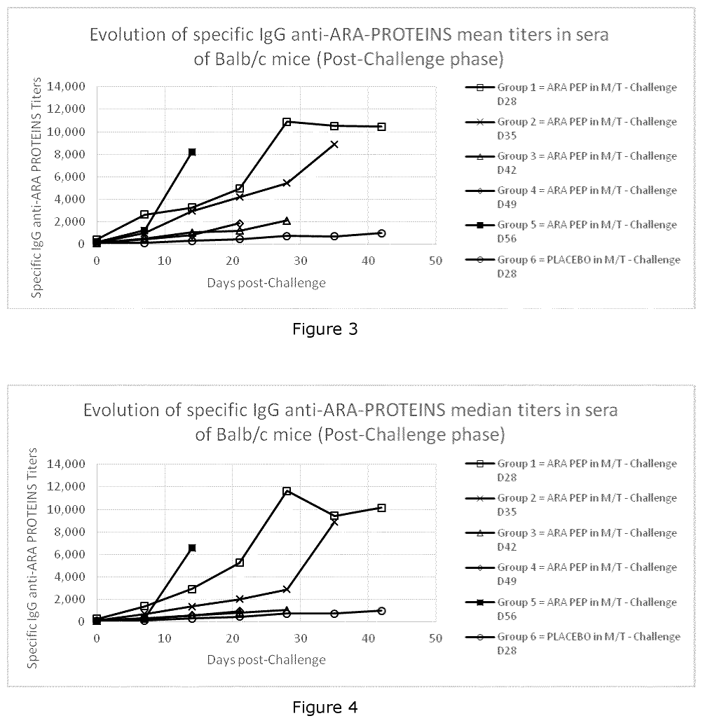

FIGS. 3 and 4 show the production of specific IgG anti-ARA PROTEINS in the sera of mice belonging to the ARA-peptides treated groups and to the Placebo-group. This production increases with different speed according to the moment when the challenge occurred. When the challenge is performed at Day 28, 35 and 56 post-beginning of the treatment, we can observe that the specific IgG anti-ARA PROTEINS production take off from Placebo-s one 2 weeks after the challenge. For the groups with the challenge occurred at Day 42 and 49 post-beginning of the treatment, no difference in the IgG production is observed to the Placebo's one till Day 21 post-challenge (and even to Day 28 post-Challenge for the D42-Challenge Group=Group 3).

For the group 1, when the Challenge is performed at Day 28 post-beginning of the treatment, a maximum in the specific IgG anti-ARA PROTEINS production seems to be reached at Day 28 post-Challenge after the going down to a plate state at D35 (and later) post-treatment.

For the group 2, when the Challenge is performed at Day 35 post-beginning of the treatment, a maximum is reached at Day 35 post-challenge but we don't have the late time-points so, we may not conclude if this point is well a maximum or if the production will increase again at late time-points.

The higher titers values of each group are the following ones:

| | Specific IgG anti-ARA PROTEINS |
|---|---|
| Days Post- | MEAN/MEDIAN TITERS |
| Challenge | Maximum value Titers |
| Group 1 | D 28 = 10.892/11.653 |
| Group 2 | D 35 = 8.900/8.894 |
| Group 3 | D 28 = 2.103/1.022 |
| Group 4 | D 21 = 1.849/947 |
| Group 5 | D 14 = 8.171/6.543 |
| Group 6 | D 42 = 990/990 |

### 6. Conclusions

From all these results, we can conclude the following points:
The treatment with ARA-peptides as described in this protocol induces a very weak production of specific IgG anti-ARA PROTEINS till Day 28 (post-beginning of the treatment)
No specific IgG production is observed in Placebo groups during the treatment phase till Day 28 (post-beginning of the treatment)
The challenge with a unique dose of 100 μg of ARA native proteins potentializes the effect of the treatment allowing an induction of the production of specific IgG anti-ARA PROTEINS reaching a maximum 4 weeks after the challenge for the group challenged at Day 28 post-beginning of the treatment. This effect is not observed in the Placebo-groups that received also the same challenge. So, we can conclude that the challenge is not sufficient per se to induce the IgG production 4 weeks after the challenge (as observed in Placebo-groups) but allows the expression of the ARA-peptides treatment. Obviously, the ARA-peptides treatment can prime the immune system allowing a “memory effect” after a challenge with native proteins.
The amplitude of the IgG response depends on the moment separating the end of the treatment and the challenge. The response is higher groups challenged at Day 28, 35 and 56 post-beginning of the treatment. Surprisingly, the groups challenged at Day 42 and 49 post-beginning of the treatment seem not to show a significative difference than the placebo-group. Maybe the production is delayed in these groups . . . . We could answer that question when we will have tested the late time-points.
All these results are related to ARA native proteins that are the closest proteins to natural allergens.

### EXAMPLE 2—HOUSE DUST MITE

Evaluation of the induction of a possible “memory effect” in Balb/c mice treated twice per week during 3 weeks with 400 μg of house dust mite peptides (HDM/PEP_SOL UBT15H20) in mannitol-trehalose environment (the injections were performed sub-cutaneously without any adjuvant) and challenged by a unique ip injection of 100 μg of house dust mite native proteins without any adjuvant.

### 7. Summary of the Protocol

| | Group 1 | | Group 2 | | Group 3 | | Group 4 | | Group 5 | | Group 6 | | Group 7 | | Group 8 | |
|---|---|---|---|---|---|---|---|---|---|---|---|---|---|---|---|---|
| Number of animals | 15 | | 15 | | 15 | | 15 | | 10 | | 10 | | 10 | | 10 | |
| Treatment: | HDM/ | | HDM/ | | HDM/PE | | HDM/PE | | PLACEBO | | PLACEBO | | PLACEBO | | PLACEBO | |
| Antigens in M/T | PEP_SOL | | PEP_SOL | | P_SOL | | P_SOL | | | | | | | | | |
| | 15H20 | | 15H20 | | 15H20 | | 15H20 | | | | | | | | | |
| Injected dose | 400 | μg | 400 | μg | 400 | μg | 400 | μg | 400 | μg | 400 | μg | 400 | μg | 400 | μg |
| (treatment) | | | | | | | | | | | | | | | | |
| Way of administration | SC | | SC | | SC | | SC | | SC | | SC | | SC | | SC | |
| (treatment) | | | | | | | | | | | | | | | | |
| Injected volume | 300 | μl | 300 | μl | 300 | μl | 300 | μl | 300 | μl | 300 | μl | 300 | μl | 300 | μl |
| (treatment) | | | | | | | | | | | | | | | | |
| Injections calendar | J0-4-7- | | J0-4-7- | | J0-4-7- | | J0-4-7- | | J0-4-7- | | J0-4-7- | | J0-4-7-11- | | J0-4-7- | |
| (treatment) | 11-14 | | 11-14 | | 11-14 | | 11-14 | | 11-14 | | 11-14 | | 14 | | 11-14 | |
| | et 18 | | et 18 | | et 18 | | et 18 | | et 18 | | et 18 | | et 18 | | et 18 | |
| Challenge | HDM/ | | HDM/ | | HDM/ | | HDM/ | | HDM/ | | HDM/ | | HDM/ | | HDM/ | |
| | ALL_SOL | | ALL_SOL | | ALL_SOL | | ALL_SOL | | ALL_SOL | | ALL_SOL | | ALL_SOL | | ALL_SOL | |
| | NATIVES | | NATIVES | | NATIVES | | NATIVES | | NATIVES | | NATIVES | | NATIVES | | NATIVES | |
| | 13G04nat | | 13G04nat | | 13G04nat | | 13G04nat | | 13G04nat | | 13G04nat | | 13G04nat | | 13G04nat | |
| Injected dose | 100 | μg | 100 | μg | 100 | μg | 100 | μg | 100 | μg | 100 | μg | 100 | μg | 100 | μg |
| (challenge) | | | | | | | | | | | | | | | | |
| Way of administration | IP | | IP | | IP | | IP | | IP | | IP | | IP | | IP | |
| (challenge) | | | | | | | | | | | | | | | | |
| Injected volume | 200 | μl | 200 | μl | 200 | μl | 200 | μl | 200 | μl | 200 | μl | 200 | μl | 200 | μl |
| (challenge) | | | | | | | | | | | | | | | | |

| | | | | | | | | |
|---|---|---|---|---|---|---|---|---|
| Injection calendar | J28 | J35 | J42 | J49 | J28 | J35 | J42 | J49 |
| (challenge) | | | | | | | | |

### 8. Denomination of the Groups

| | |
|---|---|
| Group 1 | 15H20-DP/Challenge J28 |
| Group 2 | 15H20-DP/Challenge J35 |
| Group 3 | 15H20-DP/Challenge J42 |
| Group 4 | 15H20-DP/Challenge J49 |
| Group 5 | PLACEBO (M/T)/Challenge J28 |
| Group 6 | PLACEBO (M/T)/Challenge J35 |
| Group 7 | PLACEBO (M/T)/Challenge J42 |
| Group 8 | PLACEBO (M/T)/Challenge J49 |

### 9. Correspondence Between Days Post-Beginning of the Treatment and Days Post-Challenge

All the groups have the same calendar for the treatment phase till the Day 28

| Groups | Days post-beginning of the treatment | Days Post-Challenge |
|---|---|---|
| 1 and 5 | 28-35-42-49-56-63-70-77-84-91 | 0-7-14-21-28-35-42-49- |
| | | 56-63 |
| 2 and 6 | 35-42-49-56-63-70-77-84-91-98 | 0-7-14-21-28-35-42-49- |
| | | 56-63 |
| 3 and 7 | 42-49-56-63-70-77-84-91-98-105 | 0-7-14-21-28-35-42-49- |
| | | 56-63 |
| 4 and 8 | 49-56-63-70-77-84-91-98-105-112 | 0-7-14-21-28-35-42-49- |
| | | 56-63 |

### 10. Results

Here are the results obtained by ELISA for the production of specific IgG anti-House Dust Mite proteins (native—native proteins coating)

a. Evolution of IgG Production Before any Challenge (from Day 0 to Day 28):

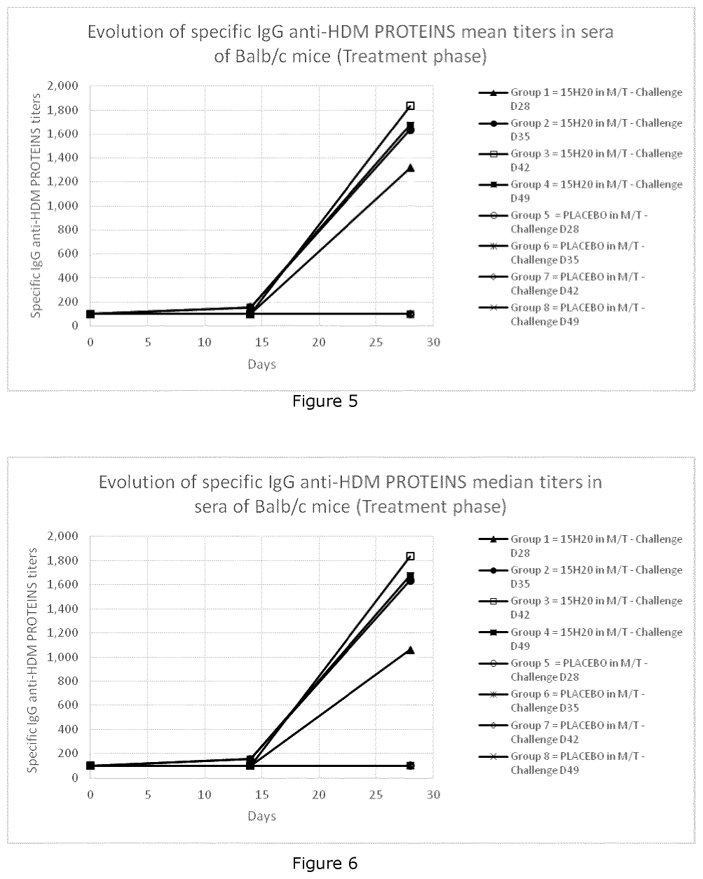

FIGS. 5 and 6 show that the treatment of the mice with house dust mite peptides as drug product leads to a weak production of specific IgG anti-HDM PROTEINS till the Day 28. The mice treated with PLACEBO show no specific IgG anti-HDM PROTEINS production (Groups 5 to 8 are at the same level)

b. Evolution of IgG Production after the Challenge (from the Challenge Day to the Day 63 Post-Challenge):

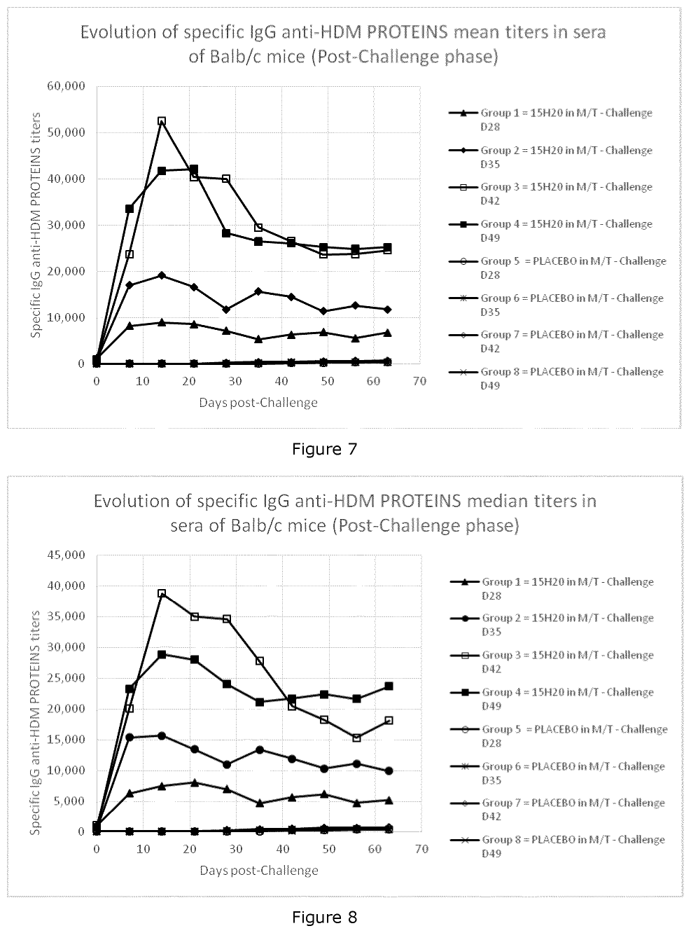

FIGS. 7 and 8 show the production of specific IgG anti-HDM PROTEINS in the sera of mice belonging to the HDM-peptides treated groups. This production increases to reach a maximum at Day 14 post-Challenge before going down to reach a relative plate state at Day 28 (for Groups 1, 2 and 4) or at Day 35 (for Group 3) post-Challenge. This production (the levels of IgG titers) seems to be linked to the moment when the Challenge occurred: the IgG productions are higher in Groups 3 and 4 (respectively challenged at Day 42 and 49 post-beginning of the treatment) than in Groups 1 and 2 (respectively challenged at Day 28 and 35 post-beginning of the treatment).

The titers values at these specific time-points are the following ones:

| Days | Specific IgG anti-HDM PROTEINS MEAN/MEDIAN TITERS | |
|---|---|---|
| Post-Challenge | Maximum value Titers | Plate State value Titers |
| Group 1 | D 14 = 9.074/7.528 | D 28 = 7.257/6.976 |
| Group 2 | D 14 = 19.203/15.715 | D 28 = 11.813/11.040 |
| Group 3 | D 14 = 52.547/38.826 | D 35 = 29.552/27.798 |
| Group 4 | D 14 = 41.817/28.886 | D 28 = 28.356/24.059 |
| Group 5 | D 63 = 458/458 | / |
| Group 6 | D 56 = 500/500 | / |
| Group 7 | D 63 = 748/748 | / |
| Group 8 | D 56 = 462/462 | / |

### 11. Conclusions

From all these results, we can conclude the following points:
The treatment with HDM-peptides as described in this protocol induces a weak production of specific IgG anti-HDM PROTEINS till Day 28 (post-beginning of the treatment)
No specific IgG production is observed in Placebo-groups during the treatment phase till Day 28 (post-beginning of the treatment)
The challenge with a unique dose of 100 μg of HDM native proteins potentializes the effect of the treatment allowing an induction of the production of specific IgG anti-HDM PROTEINS reaching a maximum 2 weeks after the challenge. This effect is not observed in the Placebo-groups that received also the same challenge. So, we can conclude that the challenge is not sufficient alone to induce the IgG production (as observed in Placebo-groups) but allows the expression of the HDM-peptides treatment. Obviously, the HDM-peptides treatment can prime the immune system allowing a “memory effect” after a challenge with native proteins.
The amplitude of the IgG response depends on the moment separating the end of the treatment and the challenge. The response is higher for late-challenged groups.
All these results are related to HDM native proteins that are the closest proteins to natural allergens.

### EXAMPLE 3—RAGWEED

Evaluation of the induction of a possible “memory effect” in Balb/c mice treated twice per week during 3 weeks with 100 μg or 400 μg of ragweed peptides (RAG/PEP_SOL UBT16E03) in mannitol-trehalose environment (the injections were performed sub-cutaneously without any adjuvant) and challenged by a unique ip injection of 100 μg of ragweed native proteins without any adjuvant.

### Summary of the Protocol

| | Groupe 1 | | Groupe 2 | | Groupe 3 | | Groupe 4 | | Groupe 5 | | Groupe 6 | | Groupe 7 | |
|---|---|---|---|---|---|---|---|---|---|---|---|---|---|---|
| Number of animals | 10 | | 10 | | 10 | | 10 | | 10 | | 10 | | 10 | |
| Treatment: | RAG/ | | RAG/ | | RAG/ | | RAG/ | | RAG/ | | RAG/ | | PLACEBO | |
| Antigens in M/T | PEP_SOL | | PEP_SOL | | PEP_SOL | | PEP_SOL | | PEP_SOL | | PEP_SOL | | | |
| | 16E03 | | 16E03 | | 16E03 | | 16E03 | | 16E03 | | 16E03 | | | |
| Injected dose | 100 | μg | 100 | μg | 100 | μg | 400 | μg | 400 | μg | 400 | μg | 100 | μg |
| (treatment) | | | | | | | | | | | | | | |
| Way of | SC | | SC | | SC | | SC | | SC | | SC | | SC | |
| administration | | | | | | | | | | | | | | |
| (treatment) | | | | | | | | | | | | | | |
| Injected volume | 200 | μl | 200 | μl | 200 | μl | 200 | μl | 200 | μl | 200 | μl | 200 | μl |
| (treatment) | | | | | | | | | | | | | | |
| Injections calendar | J0-4-7- | | J0-4-7- | | J0-4-7- | | J0-4-7- | | J0-4-7- | | J0-4-7- | | J0-4-7- | |
| (treatment) | 11-14 | | 11-14 | | 11-14 | | 11-14 | | 11-14 | | 11-14 | | 11-14 | |
| | and 18 | | and 18 | | and 18 | | and 18 | | and 18 | | and 18 | | and 18 | |
| Challenge | RAG/ | | RAG/ | | RAG/ | | RAG/ | | RAG/ | | RAG/ | | RAG/ | |
| | ALL_SOL | | ALL_SOL | | ALL_SOL | | ALL_SOL | | ALL_SOL | | ALL_SOL | | ALL_SOL | |
| | NAT 16E17 | | NAT 16E17 | | NAT 16E17 | | NAT 16E17 | | NAT 16E17 | | NAT 16E17 | | NAT 16E17 | |
| Injected dose | 100 | μg | 100 | μg | 100 | μg | 100 | μg | 100 | μg | 100 | μg | 100 | μg |
| (challenge) | | | | | | | | | | | | | | |
| Way of | IP | | IP | | IP | | IP | | IP | | IP | | IP | |
| administration | | | | | | | | | | | | | | |
| (challenge) | | | | | | | | | | | | | | |
| Injected volume | 200 | μl | 200 | μl | 200 | μl | 200 | μl | 200 | μl | 200 | μl | 200 | μl |
| (challenge) | | | | | | | | | | | | | | |

| | | | | | | | |
|---|---|---|---|---|---|---|---|
| Injection calendar | J28 | J42 | J56 | J28 | J42 | J56 | J28 |
| (challenge) | | | | | | | |

### 8. Denomination of the Groups

| | |
|---|---|
| Group 1 | 16E03-DP 100 μg/Challenge D 28 |
| Group 2 | 16E03-DP 100 μg/Challenge D 42 |
| Group 3 | 16E03-DP 100 μg/Challenge D 56 |
| Group 4 | 16E03-DP 400 μg/Challenge D 28 |
| Group 5 | 16E03-DP 400 μg/Challenge D 42 |
| Group 6 | 16E03-DP 400 μg/Challenge D 56 |
| Group 7 | PLACEBO (M/T)/Challenge J28 |

### 9. Correspondence Between Days Post-Beginning of the Treatment and Days Post-Challenge

All the groups have the same calendar for the treatment phase till the Day 28

| Groups | Days post-beginning of the treatment | Days Post-Challenge |
|---|---|---|
| 1, 4 | 28-35-42-49-56-63-70-77-84-91 | 0-7-14-21-28-35-42-49- |
| and 7 | | 56-63 |
| 2 and 5 | 42-49-56-63-70-77-84-91-98-105 | 0-7-14-21-28-35-42-49- |
| | | 56-63 |
| 3 and 6 | 56-63-70-77-84-91-98-105-112-119 | 0-7-14-21-28-35-42-49- |
| | | 56-63 |

### 10. Results

Here are the results obtained by ELISA for the production of specific IgG anti-Ragweed proteins (native—native proteins coating)

a. Evolution of IgG Production Before any Challenge (from Day 0 to Day 28):

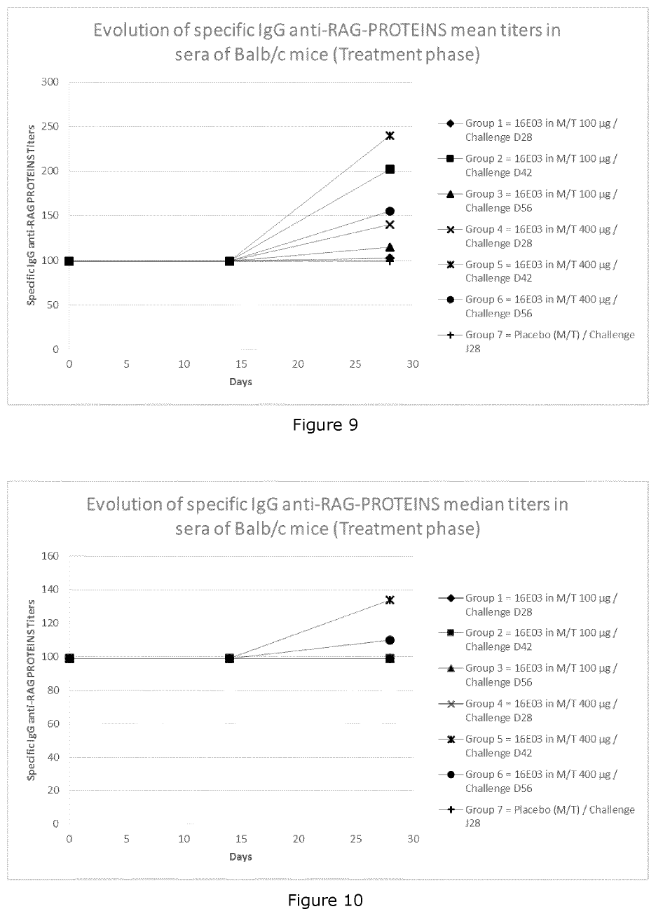

FIGS. 9 and 10 show that the treatment of mice with both concentration of ragweed peptides as drug product leads to a weak production of specific IgG anti-RAG PROTEINS until Day 28 (Group 1-6). The mice treated with PLACEBO show no production of specific IgG anti-RAG PROTEINS (Group 7)

b. Evolution of IgG Production after the Challenge (from the Challenge Day to Day 56 Post-Challenge):

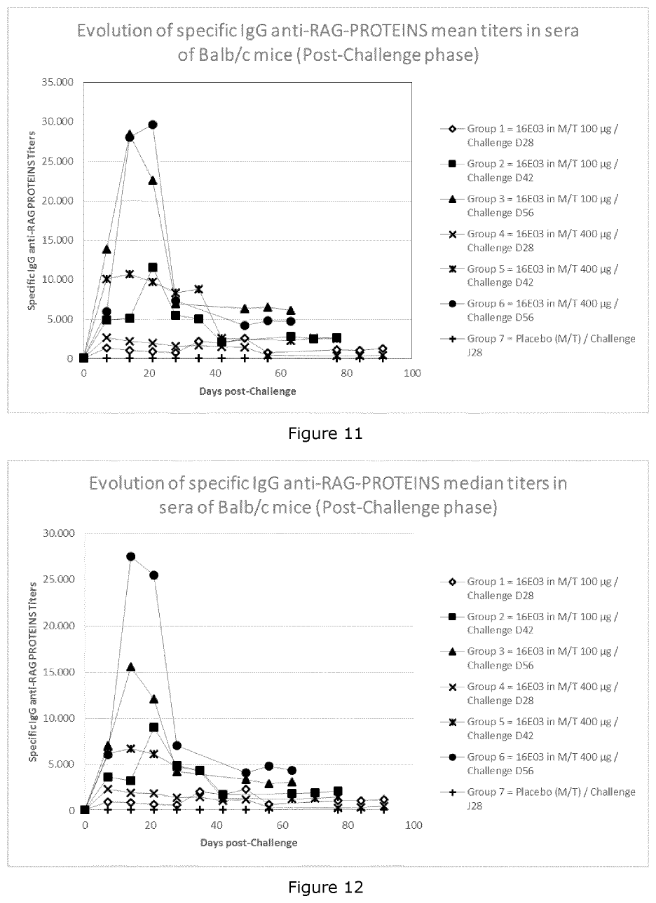

FIGS. 11 and 12 show the production of specific IgG anti-RAG PROTEINS in the sera of mice belonging to the RAG-peptides treated groups. This production increases to reach a maximum at Day 14-21 post-Challenge before going down to reach a relative plate state at Day 28-42. This production (the levels of IgG titers) seems to be linked to the moment when the Challenge occurred: the IgG productions are higher in Groups 3 and 6 (respectively challenged at Day 56 post-beginning of the treatment) than in Groups 1; 2; 4 and 5 (respectively challenged at Day 28 and Day 42 post-beginning of the treatment).

The titers values at these specific time-points are the following ones:

| | |
|---|---|
| | Specific IgG anti-RAG PROTEINS |
| Days Post- | MEAN/MEDIAN TITERS |

| Challenge | Maximum value Titers | Plate State value Titers |
|---|---|---|
| Group 1 | D 49 = 2.604/2.319 | D 56 = 799/722 |
| Group 2 | D 21 = 11.569/9.041 | D 42 = 2.137/1.720 |
| Group 3 | D 14 = 28.382/15.552 | D 28 = 6.931/4.226 |
| Group 4 | D 7 = 2.655/2.341 | D 56 = 443/351 |
| Group 5 | D 14 = 10.744/6.699 | D 42 = 2.620/1.361 |
| Group 6 | D 21 = 29.673/27.507 | D 28 = 7.352/7.042 |
| Group 7 | D 63 = 99/99 | D 28 = 99/99 |

### 11. Conclusions

From all these results, we can conclude the following points:
The treatment with RAG-peptides as described in this protocol induces a weak basal production of specific IgG anti-RAG PROTEINS till Day 28 (post-beginning of the treatment)
No specific IgG production is observed in Placebo-groups during all the study (post-treatment and post-challenge)
The challenge with a unique dose of 100 μg of RAG native proteins potentializes the effect of the treatment allowing an induction of the production of specific IgG anti-RAG PROTEINS reaching a maximum 2 weeks after the challenge. This effect is not observed in the Placebo-groups that received also the same challenge. So, we can conclude that the challenge is not sufficient alone to induce the IgG production (as observed in Placebo-groups) but allows the expression of the RAG-peptides treatment effect. In conclusion, the RAG-peptides treatment can prime the immune system allowing a “memory effect” after a challenge with native proteins.
The amplitude of the IgG response depends on the delay between the end of the treatment and the challenge. The response is higher for late-challenged groups.
The amplitude of the IgG response after challenge is dependent of the dose of the treatment with a higher response at 400 μg than at 100 μg when the challenge is performed at Day 28 and Day 42. When the challenge is performed later (Day 56) the IgG responses are similar both dosages, probably due to the amplitude of the response.
All these results are related to RAG native proteins that are the closest proteins to natural allergens.

1. A preparation comprising peptides of a natural allergen wherein the peptides are hydrolyzed allergen peptides for use in the prevention of development of allergy, wherein the preparation is administered prior to the development of any allergy against the allergen used for the preparation.

2. The preparation for use according to claim 1 wherein the preparation is administered 2 to 10 times.

3. The preparation for use according to claim 2 wherein the preparation is administered at intervals of 2 to 10 days.

4. The preparation for use according to claim 2 wherein the preparation is administered in constant amounts.

5. The preparation for use according to claim 1 wherein the preparation is free of immune stimulating adjuvants.

6. The preparation for use according to claim 1 wherein said hydrolyzed allergen peptides are obtainable by
a) extracting a natural source of allergens comprising allergenic proteins to form an extract,
b) purifying of said extract to remove non-protein components to form a purified extract,
c) denaturing said purified extract to form a purified denatured extract,
d) hydrolysing the purified denatured extract to form hydrolysed allergen peptides.

7. The preparation for use according to claim 1 wherein said hydrolyzed allergen peptides are obtainable by
a) extracting a source of allergens comprising allergenic proteins to form an extract,
b) purifying the extract to remove non-protein components to form a purified extract,
c) denaturing the purified extract with a first denaturing agent to form a purified denatured extract,
d) refining the purified denatured extract to remove impurities to form a refined denatured extract,
e) denaturing the refined denatured extract with a second denaturing agent to form denatured allergen mixture, and
f) hydrolyzing the denatured allergen mixture to form the hydrolyzed allergen peptides.

8. The preparation for use according to claim 1 wherein the allergens are selected among pollen allergens, milk allergens, venom allergens, egg allergens, weed allergens, grass allergens, tree allergens, shrub allergens, flower allergens, vegetable allergens, grain allergens, fungi allergens, fruit allergens, berry allergens, nut allergens, seed allergens, bean allergens, fish allergens, shellfish allergens, seafood allergens, meat allergens, spices allergens, insect allergens, mite allergens, mould allergens, animal allergens, pigeon tick allergens, worm allergens, soft coral allergens, animal dander allergens, nematode allergens, allergens of Hevea brasiliensis.

9. The preparation for use according to claim 1, wherein denaturing is performed with a denaturing agent selected from the group consisting of chaotropic agents, reducing agents and mixtures thereof.

10. The preparation for use according to claim 1, wherein the hydrolysis is performed with an enzyme.

11. The preparation for use according to claim 6, further comprising purifying the hydrolyzed allergens to remove peptides with molecular weights above 10.000 Da and below 1.000 Da, wherein 70% of the peptides are between 10.000 Da and 1.000 Da.

12. The preparation for use according to claim 1 for use as an allergy vaccine.

13. A method of preventing the development of allergy comprising administering a preparation comprising peptides from natural allergens to a patient at risk of developing an allergy to the natural allergens.

14. The preparation for use of claim 9 wherein the denaturing agent is selected from the group consisting of urea, guanidinium chloride, dithiotreitol, thioglycerol, ß-mercaptoethanol, TCEP (tris (2-carboxyethyl) phosphine) and mixtures thereof.

15. The preparation for use of claim 10, wherein the hydrolysis is performed in the presence of a chaotropic agent and a reducing reagent.
